(19) United States
(12) Patent Application Publication
(10) Pub. No.: US 2008/0183156 A1
(43) Pub. Date: Jul. 31, 2008

(54) Closed Suction Drainage Apparatus With Drainage Tube Having Anti-Clogging and Irrigating Functions

(75) Inventor: Gyeol Yoo, Seoul (KR)

Correspondence Address: CLARK & ELBING LLP, 101 FEDERAL STREET, BOSTON, MA, 02110, US

(73) Assignee: Catholic University Industry Academy Cooperation Foundation, Seoul (KR)

(21) Appl. No.: 11/831,186

(22) Filed: Jul. 31, 2007

(30) Foreign Application Priority Data
Jan. 26, 2007 (KR) 2007-0008296

(51) Int. Cl. A61M 27/00

(52) U.S. Cl. 604/541

(57) ABSTRACT

The present invention relates to a closed suction drainage apparatus with a drainage tube having anti-clogging and irrigating functions. The closed suction drainage apparatus of the present invention have a double tube structure of the drainage tube and its connection unit. One tube of the drainage tube is used for drain blood and exudates from a dead space and the other tube is used for applying positive pressure to remove clotted substance in the drainage tube or irrigating a wound with liquid. The closed suction drainage apparatus according to the present invention can easily clear the clogged drainage tube or irrigate dead space when needed, even in the state that it is inserted in the body.

### CROSS-REFERENCE TO RELATED APPLICATIONS

This application claims benefit of Korean Patent Application 2007-0008296, filed Jan. 26, 2007.

### BACKGROUND OF THE INVENTION

1. Field of the Invention

The present invention relates generally to closed suction drainage apparatuses for draining blood or body fluid from dead space from after operation and, more particularly, to a closed suction drainage apparatus with a drainage tube having anti-clogging and irrigating functions which is constructed such that, even if the drainage tube, inserted into the body of a patient, becomes clogged, the drainage tube can be easily cleared in the state in which it is inserted in the body, and such that the wound irrigating function can be efficiently conducted.

2. Background of the Related Art

As well known to those skilled in the art, hematoma or exudates, gathered in tissues after operation, impede the healing of a wound and cause complications. Therefore, to prevent hematoma or exudates from gathering in tissues after operation, a drainage tube is inserted into the surgery area after a surgery wound has been sutured, thus draining blood or body fluid, gathered inside the surgery wound, outside the body of the patient. This drainage tube, which is used to remove blood or exudates, gathered in the surgery area after operation, outside the body of the patient, has the shape of a longitudinal tube made of flexible material.

However, the conventional drainage tube, having the basic shape of a longitudinal tube of a predetermined length, is problematic in that draining blood and body fluid cannot be efficiently conducted, because the drainage tube is easily clogged due to the coagulation of blood in the drainage tube or the high viscosity of blood.

In an effort to overcome this problem, a variety of shape of a drainage tube was proposed and has been used. The drainage tubes having a variety of shape are able to easily drain blood or exudates gathered in the surgery area, compared to the prior drainage tube having the basic tube shape. However, these drainage tubes also have a problem in that the drainage tube is easily clogged by clotted blood or necrotic substances, in which case it cannot conduct its intended function.

### SUMMARY OF THE INVENTION

Accordingly, the present invention has been made keeping in mind the above problems occurring in the prior art, and an object of the present invention is to provide a closed suction drainage apparatus with a drainage tube having anti-clogging and irrigating functions which is constructed such that, even if the drainage tube, inserted into the body of a patient, is clogged by clotted blood or necrotic substances, the drainage tube can be easily cleared even in the state in which it is inserted in the body.

Another object of the present invention is to provide a closed suction drainage apparatus which is constructed such that, when it is required to perform continuous irrigation to prevent a wound from becoming infected, irrigation liquid is supplied to the wound through an injection passage of the drainage tube, having a double tube structure, and the irrigation liquid used for irrigating the wound is discharged outside through the drainage tube by suction force, thus providing the wound irrigation function.

In order to accomplish the above objects, the present invention provides a closed suction drainage apparatus, including: a drainage tube having a double tube structure, in which a plurality of suction holes is formed through a tube wall of the drainage tube, and an injection passage for injection of air or liquid is formed at a predetermined position in the tube wall of the drainage tube in a longitudinal direction; a drainage collection container to apply negative pressure to the drainage tube for sucking blood or body fluid, gathered in a dead space of a body of a patient, such that the blood or body fluid is drained outside the body through the drainage tube; a drainage tube connection unit, having a main body part to transmit the blood or body fluid, discharged through the drainage tube, to a discharge tube connected to the drainage collection container, at least one drainage tube connection port provided at a predetermined position in the main body part, the drainage tube connection port being connected to the drainage tube, at least one injection passage connection port provided at a predetermined position in the main body part, the injection passage connection port being connected to the injection passage, at least one injection port provided at a predetermined position in the main body part to inject air or liquid into the injection passage connection port, at least one stopper removably coupled to the injection port to openably close the injection port, and a discharge tube connecting the drainage tube connection unit to the drainage collection container.

The closed suction drainage apparatus may further include a filter provided in the injection port.

The injection passage connection port may be made of metal.

The drainage tube may have one shape selected from the group of tube shapes comprising a circular cross-section shape, a flat tube shape, and a railroad track shape.

Therefore, in the case where the drainage tube is clogged, and thus the suction drainage apparatus cannot conduct its intended function, air is injected into the injection passage, which is formed through the drainage tube, in a state in which negative pressure is maintained. Then, positive pressure is applied to the end of the drainage tube, so that the drainage tube, which has been clogged by blood clot or necrotic substances, is easily cleared. As such, the present invention can efficiently remove blood or body fluid, gathered in the dead space through the drainage tube, and avoid clogging. Furthermore, in the present invention, when it is necessary to irrigate a wound to prevent infection, irrigation liquid is supplied through the injection passage of the drainage tube having the double tube structure, thus irrigating the wound. The irrigation liquid used for irrigating the wound is discharged outside through the drainage tube by suction force. As such, the present invention has another advantage in that the wound irrigation function can be efficiently conducted.

### BRIEF DESCRIPTION OF THE DRAWINGS

The above and other objects, features and advantages of the present invention will be more clearly understood from the following detailed description taken in conjunction with the accompanying drawings, in which:

FIGS. 1 through 4 are views illustrating a closed suction drainage apparatus with a drainage tube having anti-clogging and irrigating functions, according to a preferred embodiment of the present invention, in which:

FIG. 1 illustrates the drainage tube;

FIG. 2 illustrates a drainage tube connection unit; and

FIGS. 3 and 4 illustrate representative examples of a drainage collection container which can create negative pressure.

### DETAILED DESCRIPTION OF THE PREFERRED EMBODIMENT

Hereinafter, a preferred embodiment of the present invention will be described in detail with reference to the attached drawings.

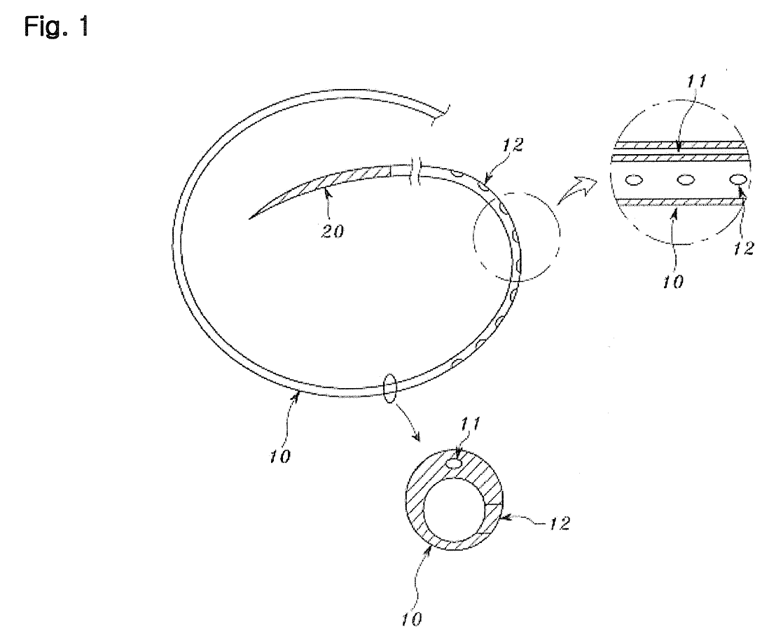

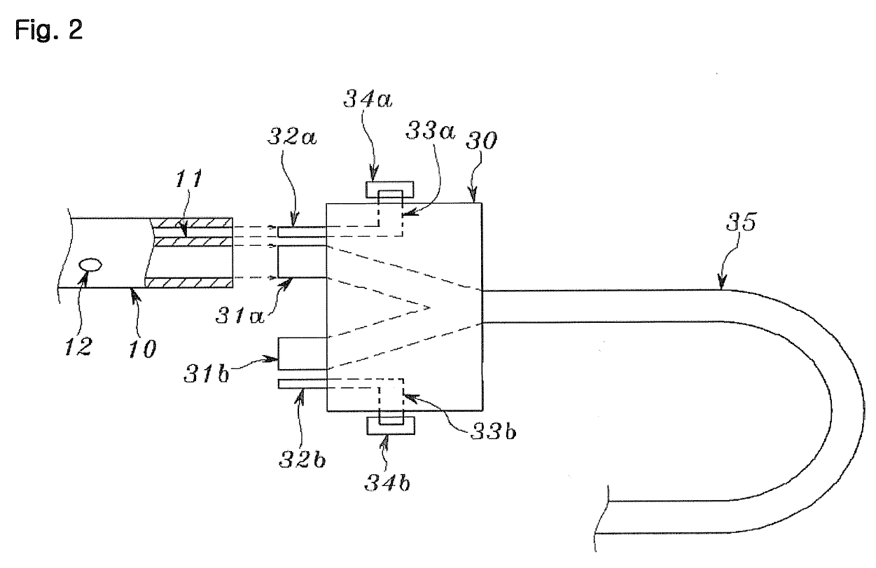

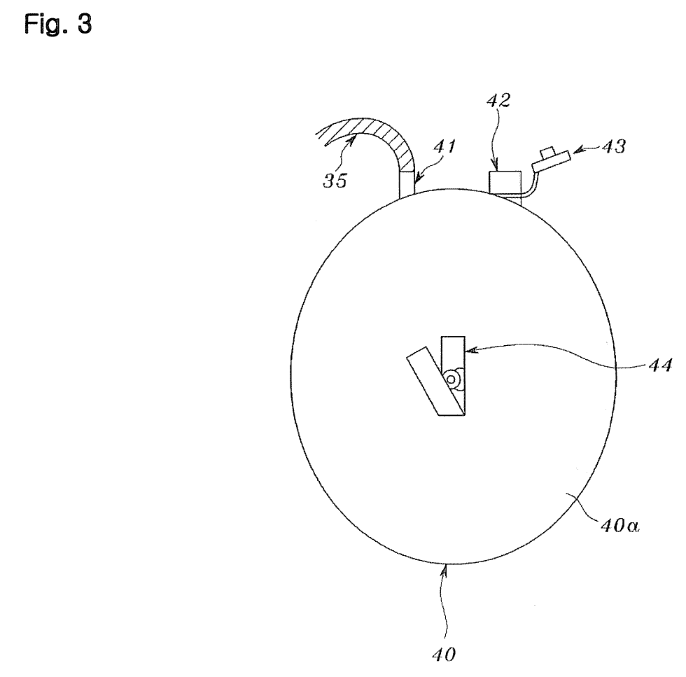

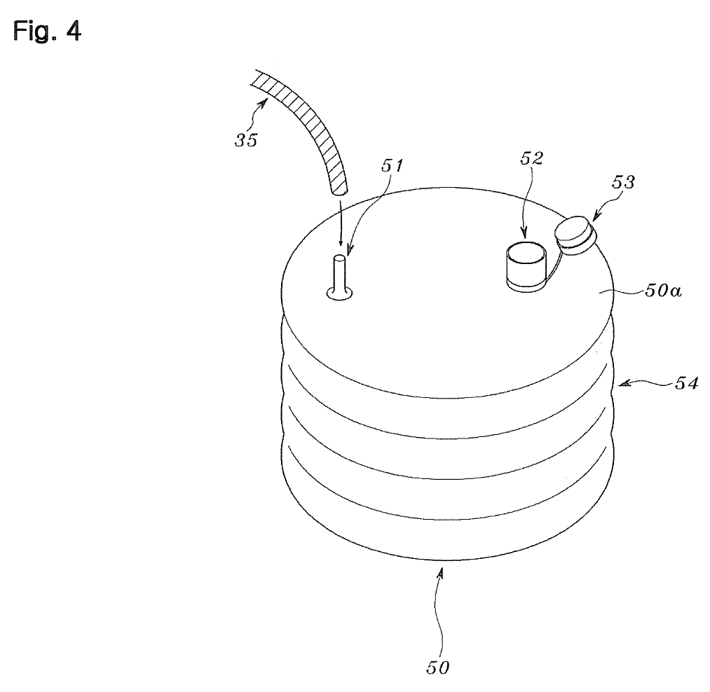

FIGS. 1 through 4 are views illustrating a closed suction drainage apparatus with a drainage tube having anti-clogging and irrigating functions, according to the preferred embodiment of the present invention. In detail, FIG. 1 illustrates the drainage tube 10, FIG. 2 illustrates a drainage tube connection unit 30, and FIGS. 3 and 4 illustrate representative examples of a drainage collection container 40, 50 which can create negative pressure.

As shown in FIG. 1, the drainage tube 10 has a double tube structure, in which a plurality of suction holes 12 is formed through a tube wall of the drainage tube 10 and an injection passage 11 for injecting air or liquid is integrally formed at a predetermined position in the tube wall in a longitudinal direction.

The drainage tube 10 is inserted into a dead space in the body of a patient, and serves as a passage for draining blood or body fluid outside the body. The suction holes 12 are formed through the tube wall of the drainage tube 10, so that blood or body fluid that enters the drainage tube 10 through the suction holes 12 is drained outside through the drainage tube 10. Furthermore, the drainage tube 10 is temporarily coupled at one end thereof to a steel needle 20 that pierces the skin of the patient to insert the drainage tube 10 into the body of the patient such that the end of the drainage tube 10 extends outside the body of the patient while part of the drainage tube 10 is placed in the body. After the drainage tube 10 has been inserted into the body of the patient by piercing the skin of the patient, the steel needle 20 is removed from the drainage tube 10, and the drainage tube 10 is connected to a drainage tube connection port 31 of the drainage tube connection unit 30.

Meanwhile, as for the size of a commonly used drainage tube 10, even if there are a variety of size, in the case of a relatively thick tube, it has an outer diameter of 4.8 mm and an inner diameter ranging from 2.8 mm to 3.2 mm, and, in the case of a relatively thin tube, it has an outer diameter of 3.2 mm and an inner diameter of 1.6 mm. Furthermore, the drainage tube 10 has one shape selected from the group of tube shapes comprising a circular cross-section shape, a flat tube shape, and a railroad track shape.

Furthermore, as shown in the sectional view of FIG. 1, the injection passage 11 is longitudinally formed through the wall of the drainage tube 10. The injection passage 11 conducts a function such that, when the drainage tube 10 becomes clogged with blood clot or necrotic substances, the blood clot or necrotic substances can be easily removed from the drainage tube 10 by injecting air through the injection passage 11. In addition, the injection passage 11 serves as a passage through which a saline solution for irrigating a wound is injected. Here, the injected saline solution is discharged outside through the drainage tube 10.

As shown in FIG. 2, the drainage tube connection unit 30 includes a main body part 30a, through which blood or body fluid drained through the drainage tube 10 is transmitted to a discharge tube 35, which is connected to the drainage collection container. Furthermore, a drainage tube connection port 31a for connection of the drainage tube 10, an injection passage connection port 32a for connection of the injection passage 11, an injection port 33a for the supply of air or liquid into the injection passage connection port 32a, and a stopper 34a for openably closing the injection port 33a are provided in respective predetermined positions in the main body part 30a. Here, the number of each of the drainage tube connection port 31a, the injection passage connection port 32a, the injection port 33a and the stopper 34a may vary as necessary. In this embodiment, the drainage tube connection unit 30 includes two drainage tube connection ports 31a and 31b, two injection passage connection ports 32a and 32b, two injection ports 33a and 33b, and two stoppers 34a and 34b, as shown in FIG. 2. For example, in the case where the surgery area is relatively large so that two drainage tubes 10 are used, the two drainage tubes 10 are connected to the drainage tube connection ports 31a and 31b, respectively. If only one drainage tube 10 is used, it is connected to one drainage tube connection port 31a or 31b, and the remaining drainage tube connection port 31a or 31b is closed.

Meanwhile, preferably, the injection passage connection port 32a, 32b for connection of the injection passage 11 is made of a steel pipe. The injection passage connection port 32a, 32b communicates with the injection port 33a, 33b through the main body part 30a. In addition, a filter is provided in the injection port 33a, 33b to prevent bacterial infection caused by the injection of air.

The drainage collection container 40, 50 is a suction bag or the like, and applies negative pressure to the drainage tube 10 to suck blood or body fluid, which is gathered in the dead space of the body of the patient, such that the blood or body fluid is drained outside the body through the drainage tube 10.

The drainage collection container 40, 50 is not limited to any special suction bag structure, so long as it can create negative pressure. In this embodiment, the circular drainage collection container 40, which is made of elastic material, and the bellows-shaped drainage collection container 50, which creates negative pressure using elastic force in a manner similar to the operation of a spring, will be explained as representative examples of the drainage collection container of the present invention.

First, as shown in FIG. 3, the circular drainage collection container 40, made of elastic material, includes a container body 40a, which receives blood or body fluid, discharged through the discharge tube 35, in a receiving chamber defined therein and is made of elastic material such that negative pressure can be created using elastic force. The circular drainage collection container 40 further includes a discharge tube connection port 41, which is provided on the container body 40a, and to which the discharge tube 35 is connected, a drainage drain port 42, which is provided in the container body 40a so that the blood or body fluid received in the receiving chamber is drained outside through the drainage drain port 42, and a stopper 43, which openably closes the drainage drain port 42.

Meanwhile, as shown in FIG. 4, the bellows-shaped drainage collection container 50 includes a container body 50a, which receives blood or body fluid, discharged through the discharge tube 35, in a receiving chamber defined therein, and has a bellows shape such that negative pressure can be created using elastic force in a manner similar to the operation of a spring. The bellows-shaped drainage collection container 50 further includes a discharge tube connection port 51, which is provided in the container body 50a, and to which the discharge tube 35 is connected, a drainage drain port 52, which is provided in the container body 50a so that the blood or body fluid received in the receiving chamber is drained outside through the drainage drain port 52, and a stopper 53, which openably closes the drainage drain port 52.

The operation of the suction drainage apparatus according to the present invention having the above-mentioned construction will be described herein below.

While blood or body fluid, gathered in dead space in the body, is sucked by negative pressure applied from the drainage collection container 40, 50 into the drainage tube 10, if the drainage tube 10 becomes clogged with coagulated blood or necrotic substances, it may not be easily cleared despite the continuous application of negative pressure. In this case, air is injected into the injection port 33a of the drainage tube connection unit 30 using an injector or the like. Then, positive pressure is applied to the end of the drainage tube 10, which is in the dead space, by air supplied through the injection passage 11. Thereby, blood clot or necrotic substances are easily removed from the drainage tube 10, to which negative pressure is continuously applied, thus the drainage tube 10, which has clogged, is cleared.

Furthermore, when the dead space must be continuously irrigated to prevent infection, a saline solution is supplied through the injection passage 11 to irrigate a wound. The saline solution, used for irrigating the wound, is discharged outside through the drainage tube 10 by suction force. As such, the present invention can conduct the wound irrigation function.

Although the preferred embodiment of the present invention has been disclosed for illustrative purposes, those skilled in the art will appreciate that various modifications, additions and substitutions are possible, without departing from the scope and spirit of the invention as disclosed in the accompanying claims.

As described above, the present invention provides a closed suction drainage apparatus with a drainage tube having anti-clogging and irrigating functions. In the present invention, in the case where the drainage tube is clogged, and thus the suction drainage apparatus cannot conduct its intended function, air is injected into an injection passage, which is formed through the drainage tube, in a state in which negative pressure is maintained. Then, positive pressure is applied to the end of the drainage tube, so that the drainage tube, which has been clogged by blood clot or necrotic substances, is easily cleared. Therefore, the present invention can efficiently remove blood or body fluid, gathered in the dead space through the drainage tube, and avoid clogging.

Furthermore, in the present invention, when it is necessary to irrigate a wound to prevent infection, irrigation liquid is supplied through the injection passage of the drainage tube having the double tube structure, thus irrigating the wound. The irrigation liquid used for irrigating the wound is discharged outside through the drainage tube by suction force. As such, the present invention has another advantage in that the wound irrigation function can be efficiently conducted.

What is claimed is:

1. A closed suction drainage apparatus, comprising:
a drainage tube having a double tube structure, in which a plurality of suction holes is formed through a tube wall of the drainage tube, and an injection passage for injection of air or liquid is formed at a predetermined position in the tube wall in a longitudinal direction;
a drainage collection container to apply negative pressure to the drainage tube for sucking blood or body fluid, gathered in a dead space of a body of a patient, such that the blood or body fluid is drained outside the body through the drainage tube; and
a drainage tube connection unit, comprising: a main body part to transmit the blood or body fluid, discharged through the drainage tube, to a discharge tube connected to the drainage collection container; at least one drainage tube connection port provided at a predetermined position in the main body part, the drainage tube connection port being connected to the drainage tube; at least one injection passage connection port provided at a predetermined position in the main body part, the injection passage connection port being connected to the injection passage; at least one injection port provided at a predetermined position in the main body part to inject air or liquid into the injection passage connection port; at least one stopper removably coupled to the injection port to openably close the injection port, and a discharge tube connecting the drainage tube connection unit to the drainage collection container.

2. The closed suction drainage apparatus as set forth in claim 1, further comprising:
a filter provided in the injection port.

3. The closed suction drainage apparatus as set forth in claim 1, wherein the injection passage connection port is made of metal.

4. The closed suction drainage apparatus as set forth in claim 1, wherein the drainage tube has one shape selected from the group of shapes comprising a circular cross-section shape, a flat tube shape and a railroad track shape.
